(19) United States
(12) Patent Application Publication
(10) Pub. No.: US 2016/0030550 A1
(43) Pub. Date: Feb. 4, 2016

(54) COMPOSITIONS, VECTORS, KITS, AND METHODS FOR IMMUNIZING AGAINST AVIAN INFECTIOUS BRONCHITIS VIRUS

(71) Applicants: Auburn University, Auburn, AL (US); The United States of America, as Represented by the Secretary of Agriculture, Washington, DC (US)

(72) Inventors: Haroldo Enrique Toro Guzman, Auburn, AL (US); Qingzhong Yu, Athens, GA (US)

(73) Assignees: AUBURN UNIVERSITY, Auburn, AL (US); The United States of America, as Represented by the Secretary of Agriculture, Washington, DC (US)

(21) Appl. No.: 14/886,757

(22) Filed: Oct. 19, 2015

Related U.S. Application Data
(63) Continuation of application No. 13/837,328, filed on Mar. 15, 2013.
(60) Provisional application No. 61/727,390, filed on Nov. 16, 2012.

(51) Int. Cl. A61K 39/215; C12N 7/00; A61K 39/17

(52) U.S. Cl. CPC A61K 39/215; A61K 39/17; C12N 7/00; A61K 2039/552

(57) ABSTRACT

Disclosed are compositions, vectors, kits, and methods for inducing an immune response against avian infectious bronchitis virus (IBV). In particular, the compositions, vectors, kits, and methods may be utilized to immunize poultry against disease associated with IBV infection or to protect poultry from IBV infection altogether.

### CROSS-REFERENCE TO RELATED APPLICATIONS

This application claims the benefit under 35 U.S.C. §119(e) to U.S. Provisional Application No. 61/727,390, filed on Nov. 16, 2012, the content of which is incorporated herein by reference in its entirety.

### BACKGROUND

The present invention relates generally to the field of compositions, vectors, kits, and methods for immunizing against coronaviruses. In particular, the invention relates to compositions, vectors, kits, and methods for immunizing avian against infection by infectious bronchitis (IB) virus (IBV).

In the poultry industry avian infectious bronchitis (IB) coronavirus (IBV) continues to be the most common contributor to respiratory disease in chicken populations despite worldwide extensive vaccination with a multiplicity of type-specific vaccines. IBV replicates primarily in the upper respiratory tract causing respiratory disease in large chicken populations. IBV's surface (S) glycoprotein is post-translationally cleaved into a S1 subunit (˜550 amino acids) and a S2 subunit (˜600 amino acids) (Lai and Holmes, 2001). Like other coronaviruses, the S1 subunit of the S glycoprotein is responsible for viral attachment to cells and is important for host protective immune responses as it induces virus neutralizing-antibodies (Cavanagh, 1981, 1983, 1984; Cavanagh and Davis, 1986; Koch et al., 1990, Koch and Kant, 1990; Mockett el al., 1984). Because of the relevance of S1 for the first step of replication (i.e., attachment to cells) and immunological escape, the extensive variation exhibited by the S1 glycoprotein among IBV coronaviruses (Kusters et al., 1987; Kusters et al., 1989b) is likely the most relevant phenotypic characteristic for this virus's “adaptation” and evolutionary success (Toro et al., 2012b). Genetic diversity among coronaviruses is achieved by high mutation frequency and recombination events (Enjuanes et al., 2000a; Enjuanes et al., 2000b; Lai and Cavanagh, 1997; Stadler et al., 2003). Selection acting on diverse populations results in rapid evolution of the virus and the emergence of antigenically different strains (Toro et al., 2012b). More than 30 different IBV types have been identified during the last 5 decades in the U.S. alone. According to a 2012 review, over 50 different genotypes of IBV are currently affecting chicken populations worldwide (Jackwood, 2012). Multiple recent surveillance studies performed in the U.S. have demonstrated that serotypes/genotypes Arkansas (Ark), Massachusetts (Mass), Connecticut (Conn), DE072, Georgia variants GAV and GA98 are currently the most prevalent (Jackwood et al., 2005; Nix et al., 2000; Toro et al., 2006).

Because IBV exists as multiple different serotypes that do not provide for cross-protection after host exposure, a multiplicity of serotype-specific IBV vaccines have been developed worldwide. For example, vaccination programs in the U.S. currently comprise mono- or polyvalent vaccines including Mass, Conn, GA98, DE072, and Ark serotypes. In Europe, IBV vaccines commonly include strains belonging to serotypes UK4/91, D274, and D-1466. However, IBV's high ability to evolve allows it to consistently circulate in commercial poultry and cause outbreaks of disease in spite of extensive vaccination. In addition, accumulating evidence indicates that attenuated IBV vaccines may also be contributing to the emergence and circulation of vaccine-like viruses in host populations (Toro ei al., 2012b; Toro et al., 2012c). Indeed, viral sub-populations differing from the predominant live vaccine population have been shown to emerge during a single passage of attenuated IBV vaccine in chickens (McKinley et al., 2008; van Santen and Toro, 2008).

In an effort to understand the mechanisms underlying the emergence of vaccine-like viruses, S1 gene sequences of virus populations of all four commercially available IBV Ark-serotype attenuated vaccines were analyzed before and after replication in chickens (Gallardo et al., 2010; van Santen and Toro, 2008). The results from these analyses demonstrated different degrees of genetic heterogeneity among Ark-derived vaccines prior to inoculation into chickens, ranging from no apparent heterogeneity to heterogeneity in 20 positions in the S gene. In all except one position, nucleotide differences resulted in different amino acids encoded and therefore in phenotypic differences among subpopulations present in the vaccines. Significantly, it has been observed that specific minor subpopulations present in each of the vaccines were rapidly “selected” during a single passage in chickens. Indeed, by 3-days post-ocular vaccination, viral populations with S gene sequences distinct from the vaccine major consensus sequence at 5 to 11 codons were found to predominate in chickens (Gallardo et al., 2010; McKinley et al., 2008; van Santen and Toro, 2008). Thus, the use of attenuated coronavirus vaccines may be contributing to the problem of antigenic variation, and the development of a novel vaccine technology to increase the resistance of chicken populations to IBV and reduce economic losses is essential for the poultry industry.

### SUMMARY

Disclosed are compositions, vectors, kits, and methods for inducing an immune response against avian infectious bronchitis virus (IBV). In particular, the compositions, vectors, kits, and methods may be utilized to immunize poultry against disease associated with IBV infection or to protect poultry from IBV infection altogether.

In the disclosed methods for immunizing an avian against infectious bronchitis virus (IBV), the method may include administering to the avian a first composition comprising a viral vector in order to prime an immune response against an IBV antigen expressed by the viral vector, and administering to the avian a second composition comprising IBV (e.g., to boost the immune response against the antigen). The IBV antigen typically is the S2 polypeptide or a variant thereof. The second composition may be administered about 1, 2, 3, 4, 5, 6 weeks or more, subsequent to administering the first composition.

Typically in the methods, the first composition is administered prior to the second composition in order to prime an immune response against an IBV antigen. However, it is contemplated herein that the first composition and second composition might be administered concurrently or that the second composition might be administered prior to the first composition.

In the methods, the first composition and the second composition typically are administered in an amount that is effective for inducing an immune response against one or more proteins of IBV, and in particular, the S2 polypeptide or a variant thereof. The induced immune response may include an antibody response (i.e., a humoral response), a cell-mediated response, or both.

In the methods, the first composition typically comprises a viral vector that expresses the S2 polypeptide or a variant thereof. Suitable viral vectors may include, but are not limited to, a paramyxovirus vector, an adenovirus vector, a herpesvirus vector, a retrovirus vector, and a poxvirus vector. In particular, suitable paramyxovinrs vectors may include recombinant Newcastle disease virus vectors (rNDV) such as the recombinant LaSota vector (rLS) where the S2 polypeptide or a variant thereof is inserted, for example, between the phosphoprotein gene and the matrix gene.

In the methods, the second composition typically comprises IBV in a form suitable for boosting the immune response that was primed by administering the first composition. The IBV may be an attenuated form, or an inactivated form or IBV (preferably an attenuated form).

The disclosed methods typically are practiced on avians. Suitable avians may include birds such as poultry and in particular chickens.

Also disclosed herein are compositions (e.g., vaccine compositions), vectors, and kits for practicing the disclosed methods. The composition, vectors, and kits may comprise or provide components or agents for immunizing an avian against infectious bronchitis virus (IBV) or for protecting an avian from IBV infection altogether.

### BRIEF DESCRIPTION OF THE DRAWINGS

FIG. 1 provides a schematic representation of construction of the pLS/IBV-S2 recombinant expression vector for the synthetic S2 transgene.

FIG. 2 illustrates the detection of the pLS/IBV-S2 recombinant expression vector by RT-PCR.

FIG. 3 illustrates sequencing analysis of the pLS/IBV-S2 recombinant expression vector.

FIG. 4 graphically illustrates respiratory signs (tracheal and nasal rales) detected in chickens that were challenged with a virulent IBV Arkansas (Ark)-type strain after having been administered a rLS.IBVS2 Mass-type prime/boost vaccination regimen described herein. Challenged control groups included chickens vaccinated with the empty vector (NDVE)+Mass, and chickens vaccinated only with the empty vector. An additional group (NN) was unvaccinated/not challenged. Signs were assessed blindly. Different letters (a, b, c) indicate significant differences (P<0.05).

FIG. 5 illustrates IBV RNA quantification in tears of chickens that were administered the prime/boost vaccination regimen described in FIG. 4. Controls included chickens vaccinated with the rLS empty vector (rLS/E)+Mass, and chickens vaccinated with rLS/E only. Different letters (a, b, c) indicate significant differences (P<0.05). Viral RNA determined 4 d post-challenge was significantly reduced in chickens primed with LS/IBV.S2 and boosted with Mass.

### DETAILED DESCRIPTION

Disclosed are compositions, vectors, kits, and methods for inducing an immune response against avian infectious bronchitis virus (IBV) which may be described herein using definitions as set forth below and throughout the application.

Unless otherwise specified or indicated by context, the terms “a,” “an,” and “the,” mean “one or more.” For example, “an antigen” should be interpreted to mean “one or more antigens.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “subject,” “host,” or “individual” typically refer to an avian at risk for acquiring an infection by infectious bronchitis virus (IBV). The terms “subject,” “host,” or “individual” may be used interchangeably. Suitable avians for the disclosed methods and kits may include poultry such as members of the order Galliformes, and in particular the species Gallus gallus or the subspecies Gallus gallus domesticus.

As used herein “IBV” refers to “avian bronchitis virus” which is a coronavirus that infects chicken and causes the associated disease “IB.” The term “IBV” is meant to encompass numerous serotypes of IBV which have been isolated and characterized including: B/D207/84: B/D274/84; B/UK167/84; B/UK142/86; E/D3896/84; E/UK123/82; Brazil/BRI/USP-73/09; 79318/4-91/91; FR/CR88121/88; China/Q1/98; China/LDL971/97 aaz09202; CAV/CAV9437/95; CAV/CAV1686/95; CAV/CAV56b/91; PA/Wolgemuth/98; PA/171/99; CA/557/03 S1; JAA/04 S1 vaccine; HN99 S1; N1/62/S1; GA08 S1 GU301925; Ark/ArkDPI/81 S1; Ark/Ark99/73; CAL99/CAL99/99 S1; CAL99/NE15172/95 S1; Holte/Holte/54; JMK/JMK/64; Gray/Gray/60; Iowa/Iowa609/56; Ca/1737/04 S1; DMA/5642/06 S1; GA07/GA07/07 S1; QX/QX1BV/99; Mass/H52 S1; Mass/H120 S1; Mass/Mass41/41 S1; Conn/Conn46/51 S1 vaccine; FL/FL18288/71; DE/DE072/92 S1 vaccine; GA98/0470/98 S1; and Dutch/D1466/81.

The serotype of IBV is generally determined by a host's humoral immune response against the S1 polypeptide. Hence, the serotype of IBV is generally determined by the amino acid sequence of the S1 polypeptide. Because the presently disclosed methods and kits utilize the S2 polypeptide as an antigen, an avian may be vaccinated against a strain of IBV, and subsequently, the avian may be protected against a strain of IBV having a different serotype than the administered strain. Therefore, the disclosed methods may be practiced in order to induce cross-protection against different strains of IBV, which is referred to as “heterotypic protection,” whereas “homotypic protection” is protection against the administered strain of IBV. For example, in the disclosed methods, an avian may be administered a Massachusetts-type strain of IBV, and subsequently the avian may be protected against disease and/or infection by not only a Massachusetts-type strain of IBV, but also an Arkansas-type strain of IBV.

The presently disclosed methods and kits may utilize naturally occurring avirulent strains of IBV. Alternatively, the presently disclosed methods and kits may utilize live attenuated strains of IBV. Live attenuated strains of IBV are available commercially as vaccines and may include Mass/Mass41/41 S1 and Ark/ArkDPL/81 S1. The complete genomic sequence of Ark/ArkDPI/81 has been reported. (See Ammayappan et al., Virology Journal 2008, 5:157, which is incorporated herein by reference in its entirety).

As used herein, an “immune response” may include an antibody response (i.e., a humoral response), where an immunized individual is induced to produce antibodies against an administered antigen (e.g., IgY, IgA, IgM, IgG, or other antibody isotypes) and may also include a cell-mediated response, for example, a cytotoxic T-cell response against cells expressing foreign peptides derived from an administered antigen in the context of a major histocompatibility complex (MHC) class I molecule.

As used herein, “potentiating” or “enhancing” an immune response means increasing the magnitude and/or the breadth of the immune response. For example, the number of cells that recognize a particular epitope may be increased (“magnitude”) and/or the numbers of epitopes that are recognized may be increased (“breadth”).

As used herein, “viral load” is the amount of virus present in a sample from a subject infected with the virus. Viral load is also referred to as viral titer or viremia. Viral load can be measured in variety of standard ways including copy Equivalents of the viral RNA (vRNA) genome per milliliter individual sample (vRNA copy Eq/ml). This quantity may be determined by standard methods that include RT-PCR.

The terms “polynucleotide,” “nucleic acid” and “nucleic acid sequence” refer to a polymer of DNA or RNA nucleotide of genomic or synthetic origin (which may be single-stranded or double-stranded and may represent the sense or the antisense strand). The polynucleotides contemplated herein may encode and may be utilized to express one or more IBV polypeptides such as the S2 polypeptide or variant thereof.

As used herein, polypeptide, proteins, and peptides comprise polymers of amino acids, otherwise referred to as “amino acid sequences.” A polypeptide or protein is typically of length ≧100 amino acids (Garrett & Grisham, Biochemistry, 2^{nd} edition, 1999. Brooks/Cole, 110). A peptide is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). However, the terms “polypeptide,” “protein,” and “peptide” may be used interchangeably herein.

As contemplated herein, a polypeptide, protein, or peptide may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor fobrmation, hydroxylation, iodination (e.g., of thyroid hormones), and phosphorylation (e.g., the addition of a phosphate group, usually to serine, tyrosine, threonine or histidine).

The amino acid sequences contemplated herein may include substitutions related to a reference amino acid sequence. In some cases, these substitutions may be conservative amino acid substitutions relative to the reference amino acid sequence. For example, a variant, mutant, or derivative polypeptide may include conservative amino acid substitutions relative to a reference polypeptide. “Conservative amino acid substitutions” are those substitutions that are predicted to interfere least with the properties of the reference polypeptide. In other words, conservative amino acid substitutions substantially conserve the structure and the function of the reference protein. Table 1 provides a list of exemplary conservative amino acid substitutions.

TABLE 1

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The words “insertion” and “addition” refer to changes in an amino acid sequence resulting in the addition of one or more amino acid residues. For example, an insertion or addition may refer to 1, 2, 3, 4, 5, 10, 20, 30, 40, 50, 60, 70, 80, 90, 100, 150, or 200 amino acid residues.

A “deletion” refers to a change in the amino acid or nucleotide sequence that results in the absence of one or more amino acid residues. For example, a deletion may remove at least 1, 2, 3, 4, 5, 10, 20, 50, 100, or 200 amino acids residues. A deletion may include an internal deletion or a terminal deletion (e.g., an N-terminal truncation or a C-terminal truncation of a reference polypeptide).

A “fragment” is a portion of an amino acid sequence which is identical in sequence to but shorter in length than a reference sequence. A “fragment” as contemplated herein refers to a contiguous portion of an amino acid reference sequence. For example, a fragment of a polypeptide refers to less than a full-length amino acid sequence of the polypeptide (e.g., where the polypeptide is truncated at the N-terminus, the C-terminus, or both termini). A fragment may comprise up to the entire length of the reference sequence, minus at least one amino acid residue. For example, a fragment may comprise from 5 to 1000 contiguous amino acid residues of a reference polypeptide. In some embodiments, a fragment may comprise at least 5, 10, 15, 20, 25, 30, 40, 50, 60, 70, 80, 90, 100, 150, 250, or 500 contiguous amino acid residues of a reference polypeptide, respectively. Fragments may be preferentially selected from certain regions of a molecule. The term “at least a fragment” encompasses the full length polypeptide. An “immunogenic fragment” of a polypeptide is a fragment of a polypeptide typically at least 5 or 10 amino acids in length that includes one or more epitopes of the fill-length polypeptide.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “variant,” “mutant,” or “derivative” of a particular polypeptide sequence is defined as a polypeptide sequence having at least 50% sequence identity to the particular polypeptide sequence over a certain length of one of the polypeptide sequences using blastp with the “BLAST 2 Sequences” tool available at the National Center tbr Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of polypeptides may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides. A “variant” or a “derivative” may have substantially the same functional activity as a reference polypeptide. For example, a variant or derivative of the IBV S2 polypeptide may have one or more functional activities associated with the wild-type IBV S2 polypeptide including, but not limited to, interacting with the S1 polypeptide, interacting with the viral membrane of IBV, and/or facilitating fusion of IBV with a host cell membrane.

IBV S2 Glycoprotein

As used herein, “structural viral proteins” of IBV are those proteins that are physically present in the virus. The structural proteins of IBV may be utilized in the compositions, vectors, kits, and methods disclosed herein and may include the S2 polypeptide.

The amino acid sequence of IBV S2 polypeptide is disclosed herein as “SEQ ID NO:1” which is derived from the amino acid sequence referenced by accession no. AAF82269.1 at the GenBank database. S2 is a class I viral fusion protein which functions to facilitate fusion of the IBV membrane with a cellular host. The native S2 polypeptide is glycosylated to form a “glycoprotein.” The most common glycosylating groups or “glycans” are classified as N-glycans and O-glycans. In N-glycans, an amido group in a side chain of asparagine (N) is N-glycosylated. In O-glycans, an alcohol in a side chain of serine (S) or threonine (T) is glycosylated. The S2 polypeptide expressed by the vectors disclosed herein may be similarly glycosylated when the S2 polypeptide is expressed in a host.

Vectors

The term “vector” refers to some means by which DNA or RNA can be introduced into a host. There are various types of vectors including virus, plasmid, bacteriophages, cosmids, and bacteria. As used herein, a “viral vector” refers to recombinant viral nucleic acid that has been engineered to express a heterologous polypeptide (e.g., an IBV S2 polypeptide). The recombinant viral nucleic acid typically includes cis-acting elements for expression of the heterologous polypeptide. The recombinant viral nucleic acid typically is capable of being packaged into a helper virus that is capable of infecting a host cell. For example, the recombinant viral nucleic acid may include cis-acting elements for packaging. Preferably, the viral vector is not replication competent, is attenuated, or at least does not cause disease. The viral vector may be genetically altered by modern molecular biological methods (e.g., restriction endonuclease and ligase treatment, and rendered less virulent than wild type), typically by deletion of specific genes. For example, the recombinant viral nucleic acid may lack a gene essential for production of infectious or virulent virus.

The recombinant viral nucleic acid may function as a vector for an immunogenic IBV protein by virtue of the recombinant viral nucleic acid encoding foreign DNA. The recombinant viral nucleic acid, packaged in a virus (e.g., a helper virus) may be introduced into a vaccinee by standard methods for vaccination of live vaccines. A live vaccine of the invention can be administered at, for example, about 10⁴ to 10⁸ viruses/dose, or 10⁶ to 10⁹ pfu/dose. Actual dosages of such a vaccine can be readily determined by one of ordinary skill in the field of vaccine technology.

Numerous virus species can be used as the recombinant virus vectors for the composition disclosed herein. A preferred recombinant virus vector for a viral vaccine is a recombinant paramyxovirus (e.g., recombinant Newcastle disease virus (rNDV) LaSota vector (rLS). Recombinant NDV vector have been used previously to express transgenes. (Bukreyev and Collins, 2008; Bukreyev et al., 2005; DiNapoli et al., 2007; DiNapoli et al., 2009; Ge et al., 2007; Ge et al., 2010; Huang et al., 2003a; Huang et al., 2004; Nakaya et al., 2001; Nayak et al., 2009; Park et al., 2006; Swayne et al., 2003). Other suitable viral vectors may include recombinant adenovirus, herpesvirus, retrovirus, or poxvirus vectors. Coronavirus and Influenza virus transgenes have been expressed from replication-defective recombinant adenovirus, and the recombinant adenoviruses have proven to be stable and to induce strong immune responses (Toro et al., 2012a; Toro et al., 2012c; Toro et al., 2007; Toro et al., 2008).

Suitable virus species for vectors may include virus species that naturally are not virulent for chickens. Preferred virus species for vectors include lentogenic Newcastle disease strains. Such strains are naturally not virulent, pathogenic, or exhibit only reduced pathogenicity for chickens. Other vectors used in the poultry industry to vaccinate chickens include herpesvirus of turkeys (HVT). These viruses also are not naturally virulent for chickens and do not need to be modified further in order to reduce their virulence.

Codon Optimization

The transgene expressed in the vectors disclosed herein may have the native polynucleotide sequence of S2 or may have a polynucleotide sequence that has been modified. For example, the presently disclosed vectors may express polypeptides from polynucleotides that encode the polypeptides where the polynucleotides contain codons that are optimized for expression in a particular host. For example, presently disclosed vectors may include one or more polypeptides from IBV where the encoding polynucleotide sequence is optimized to include codons that are most prevalent in an avian such as a chicken. Codon usage for the chicken genome has been reported. (See Rao el al., DNA Res. 2011 December, 18(6):499-512, which is incorporated herein by reference). Accordingly, a polynucleotide encoding the amino acid sequence of SEQ ID NO:1 is contemplated herein wherein the polynucleotide's nucleic acid sequence has been codon-optimized for expressing SEQ ID NO:1 in chicken (i.e., codon-optimized based on codon usage for the chicken genome). A codon-optimized polynucleotide for expressing SEQ ID NO:1 is reported herein as SEQ ID NO:2.

Formulation of the Compositions

The compositions disclosed herein may be formulated as vaccine compositions for administration to a subject in need thereof. Such compositions can be formulated andior administered in dosages and by techniques well known to those skilled in the medical arts taking into consideration such factors as the age, sex, weight, and condition of the particular subject and the route of administration. The compositions may include carriers, diluents, or excipients as known in the art. Further, the compositions may include preservatives (e.g., anti-microbial or anti-bacterial agents such as benzalkonium chloride) or adjuvants.

The compositions may be administered prophylactically. In prophylactic administration, the vaccines may be administered in an amount sufficient to induce immune responses for protecting against IBV infection (i.e., a “vaccination effective dose” or a “prophylactically effective dose”).

The composition disclosed herein may be formulated for delivered via a variety of routes. Routes may include, but are not limited to, parenteral administration (e.g., intradermal, intramuscular or subcutaneous delivery), aerosol administration (e.g., using spray cabinets), oral administration, and intraocular administration.

Adjuvants

The disclosed compositions may include an adjuvant. The term “adjuvant” refers to a compound or mixture that enhances the immune response to an antigen. An adjuvant can serve as a tissue depot that slowly releases the antigen and also as a lymphoid system activator that non-specifically enhances the immune response. Examples of adjuvants which may be employed include MPL-TDM adjuvant (monophosphoryl Lipid A/synthetic trehalose dicorynomycolate, e.g., available from GSK Biologics). Another suitable adjuvant is the immunostimulatory adjuvant AS021/AS02 (GSK). These immunostimulatory adjuvants are formulated to give a strong T cell response and include QS-21, a saponin from Quillay saponaria, the TL4 ligand, a monophosphoryl lipid A, together in a lipid or liposomal carrier. Other adjuvants include, but are not limited to, nonionic block co-polymer adjuvants (e.g., CRL1005), aluminum phosphates (e.g., AIPO4), R-848 (a Thl-like adjuvant), imiquimod, PAM3CYS, poly (I:C), loxoribine, potentially useful human adjuvants such as BCG (bacille Calmette-Guerin) and Corynebacterium parvum, CpG oligodeoxynucleotides (ODN), cholera toxin derived antigens (e.g., CTAI-DD), lipopolysaccharide adjuvants, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions in water (e.g., MF59 available from Novartis Vaccines or Montanide ISA 720), keyhole limpet hemocyanins, and dinitrophenol.

Prime-Boost Vaccination Regimen

As used herein, a “prime-boost vaccination regimen” refers to a regimen in which a subject is administered a first composition one or more times (e.g., two or three times with about 2, 3, or 4 weeks between administrations) and then after a determined period of time (e.g., about 1 week, about 2 weeks, about 4 weeks, about 2 months, about 3 months, about 4 months, about 5 months, about 6 months, or longer), the subject is administered a second composition. The second composition may also be administered more than once, with at least 2, 3, or 4 weeks between administrations. The first and second compositions may be the same or different. For example, the first composition may include a recombinant viral vector and the second composition may include a live, attenuated virus.

Characterization of the Immune Response in Vaccinated Individuals

The compositions disclosed herein may be delivered to subjects at risk for infection with IBV. Subsequently, the efficacy of the vaccine may be assessed based on the immune response induced by administering the vaccine. In order to assess the efficacy of the vaccine, the immune response can be assessed by measuring the induction of antibodies to an antigen or particular epitopes of an antigen or by measuring a T-cell response to an antigen or particular epitopes of an antigen. Antibody responses may be measured by assays known in the art such as ELISA. T-cell responses may be measured, for example, by using tetramer staining of fresh or cultured PBMC, ELISPOT assays or by using functional cytotoxicity assays, which are well-known to those of skill in the art.

### Examples

The following examples are illustrative and are not intended to limit the disclosed subject matter.

Use of S2 Transgenes to Elicit Heterotypic Protection Against IBV Infection

Introduction

Basis for Using S2 Transgenes to Elicit Heterotypic Protection.

Unlike the S1 subunit, the S2 subunit of the S polypeptide is highly conserved among different coronavirus strains (Kusters el al., 1989a). S1 amino acid sequence identity between different serotypes reaches as low as 44.7% (Gelb el al., 1997). In contrast, an analysis of all 251 complete IBV S2 sequences available in GenBank from all over the world indicates that the percent amino acid sequence identity among them varies between 74% and 100% (data not shown). The fact S2 amino acid sequence similarity also results in antigenic similarity has been demonstrated by producing monoclonal antibodies against the S2 protein of IBV Mass serotype strain M41 (Souza et al., 2001). These antibodies recognized the homologous M41 strain but also the distant genotypic strains Ark-99, Conn, and numerous strains in South America (Souza et al., 2001). From a teleological perspective, exposing conserved regions to the immune system would be detrimental to the success of this virus family. Thus, probably due to protein folding or other mechanisms, S2 remains largely unexposed to the immune system during coronavirus infection and indeed the strongest neutralizing antibody responses elicited in chickens are directed against the S1 protein (Cavanagh et al., 1986).

Theoretically, if the S2 subunit were exposed during the natural infection process, both vaccinated and naturally infected animals would become resistant to subsequent challenge with coronaviruses that exhibit different antigenicity based on the S1 subunit's amino acid sequence. This is not observed to occur naturally because animals become re-infected with different serological strains of coronavirus in spite of having recovered from infection with a previous strain.

However, the fact that only a limited immune response is triggered by the S2 subunit does not necessarily mean that the S2 subunit is less immunogenic than the S1 subunit. Here, we tested whether overexposing the S2 subunit to the immune system by means of a vectored vaccine, followed by boosting with whole virus would result in enough memory cells with S2 subunit specificity to protect the host against diverse coronavirus variants having antigenically dissimilar S1 subunits.

Basis for the Use of Recombinant Newcastle Disease Virus Strain LaSota to Express IBV S2 Genes.

For the purpose of expressing the IBV S2 gene, a recombinant Newcastle disease virus (NDV) LaSota vector (rLS) was selected. NDV, the etiologic agent of Newcastle disease (ND), is a non-segmented, single-stranded, negative sense RNA virus that belongs to the genus Avulavirus within the Paramyxoviridae family (Lamb et al., 2005). Its genome is approximately 15.2 kb in length and encodes six major proteins including nucleoprotein (NP), phosphoprotein (P), matrix (M), fusion (F), hemagglutinin-neuraminidase (HN) and large protein (L) or polymerase in the order 3′-leader-NP-P-M-F-HN-L-trailer-5′ (Lamb ec al., 2005; Pedersen et al., 2004). Naturally-occurring low pathogenic NDV strains, such as BI and LaSota strains, are routinely used as live vaccines throughout the world for prevention of the disease in avian species (Alexander and Senne, 2008; Hitchner, 2004).

During the last decade, reverse genetics systems have been developed to genetically manipulate the genome of NDV for studying the molecular biology of the virus (Estevez el al., 2007; Estevez et al., 2011; Huang el al., 2003a; Huang et al., 2003b; Huang et al., 2004; Peeters el al., 1999; Romer-Oberdorfer et al., 1999), and to generate recombinant NDVs that express foreign proteins from added genes for development of vectored vaccines (Bukreyev and Collins, 2008; Bukreyev et al., 2005; DiNapoli et al., 2007; DiNapoli el al., 2009; Ge et al., 2007; Ge et al., 2010; Huang et al., 2003a; Huang et al., 2004; Nakaya tI al., 2001; Nayak et al., 2009; Park el al., 2006; Swayne et al., 2003), In particular, reverse genetics systems have been developed for the study of avian paramyxovirus pathogenesis, the design of an improved vaccine, and the development of a LaSota vaccine strain-based multivalent vaccine vector (Estevez et al., 2007; Estevez et al., 2011; Miller et al., 2009; Susta et al., 2010; Yu et al., 2011; Yu et al., 2010b). Several recombinant LaSota viruses expressing foreign proteins, such as the glycoprotein (G) of avian metapneumovirus subgroup C and the HA protein of avian influenza virus, have been generated and evaluated in vitro and in vivo as bivalent vaccine candidates (Bowen et al., 2010; Hlu et al., 2011; Yu et al., 2010a).

Results

Construction of a Recombinant LaSota cDNA Clone Containing the S2 Gene of IBV.

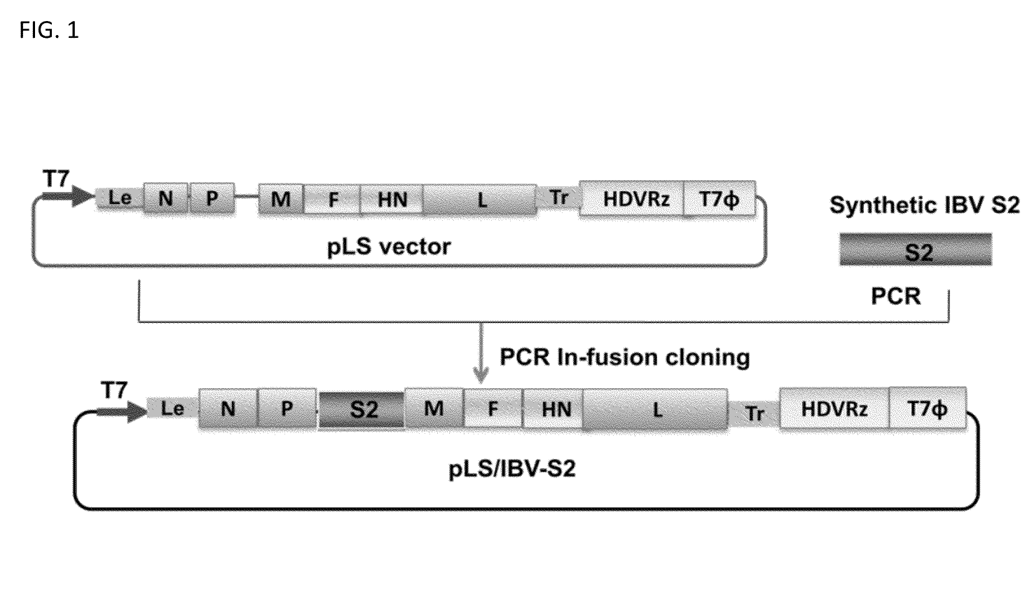

To construct a recombinant cDNA clone containing the IBV S2 gene, the previously generated full-length LaSota cDNA clone was used as a backbone. The complete S2 gene sequence (SEQ ID NO: 1) was codon-optimized for expression in chicken cells and synthesized (SEQ ID NO:2). The synthetic codon-optimized IBV S2 gene was inserted into the rNDV vector between the phosphoprotein (P) and matrix (M) genes as an additional transcription unit using the In-Fusion® PCR cloning kit (Clontech) (FIG. 1). The resulting recombinant clone, designated as pLS/IBV-S2, was amplified in Stbl2 cells and purified using a QIAprep Spin Miniprep kit (Qiagen). The sequence fidelity of the recombinant clone was confirmed by nucleotide sequencing with the Applied Biosystems-PRISM fluorescent big dye sequencing kit and the ABI 3730 DNA Sequencer. The total length of the clone obeyed the rule of six (i.e., the nucleotide length of the genome was a multiple of six), which is critical for efficient replication of the virus genome of paramyxoviruses and their vectors.

Recombinant Virus Rescue and Propagation.

Rescue of the recombinant LS/IBV-S2 virus was performed by transfecting the full-length cDNA clone and supporting plasmids into MVA/T7-infected HEp-2 cells using Lipofectamine™ 2000 (Invitrogen) according to the manufacturer's instruction. At 6 h post-transfection, the cells were washed with phosphate buffered saline (PBS) and maintained in DMEM medium containing 2% FBS and antibiotics. At 72 h post-transfection, the transfected/infected cells were harvested by freeze-thawing three times. The rescued virus was amplified by inoculating 100 μl of the transfected/infected cell lysate into the allantoic cavity of 9-d-old SPF chicken embryos. After 4 days of incubation, the allantoic fluid (AF) was harvested and used for detection of rescued virus by the hemagglutination (HA) test. The HA positive AF was terminally diluted during subsequent passages to remove any possible MVA contamination. The rescued virus, designed as rLS/IBV-S2, was amplified in SPF chicken embryos three times and the AF was harvested and stored at −80 C as a stock.

Confirmation of the Rescued rLS-S2.

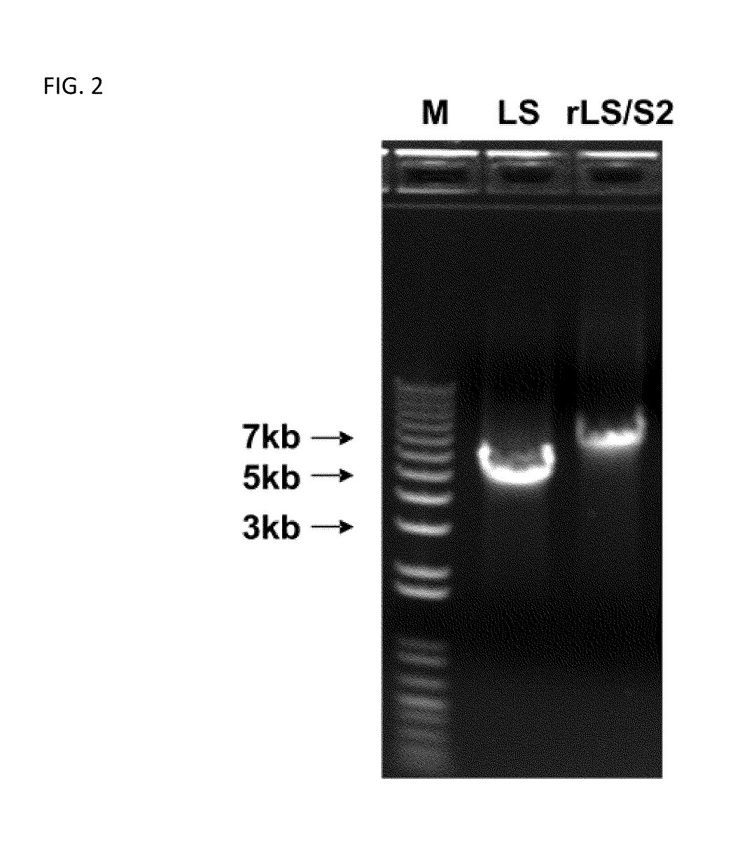

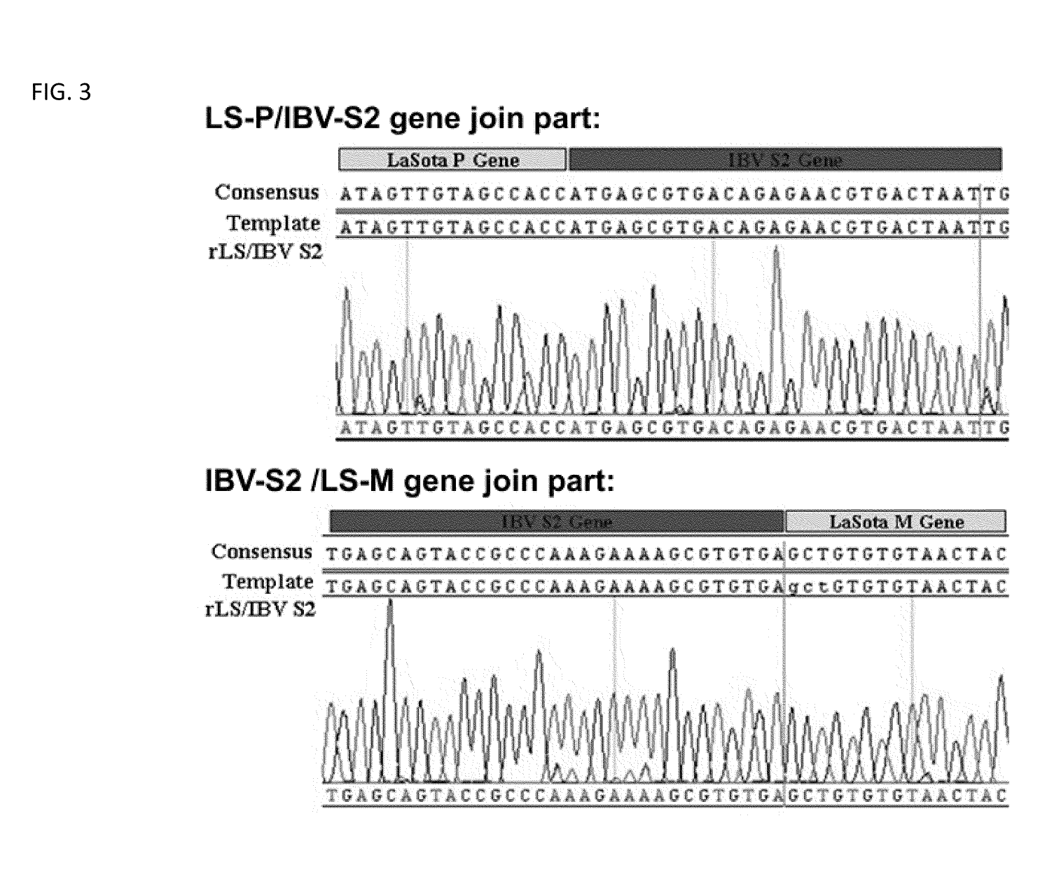

To confirm the sequence fidelity of the rescued virus, the S2 gene insertion region of rLS/IBV-S2 was examined by RT-PCR amplification with a pair of specific primers followed by sequencing analysis. The results showed that the RT-PCR product generated from the rescued rLS/IBV-S2 virus is about 2.0 kb larger than that from the parental LaSota virus (FIG. 2.). Sequencing analysis of the RT-PCR product confirmed that the synthetic IBV S2 gene has been inserted into the LaSota genome between the P and M genes (FIG. 3). The complete genome of the rescued rLS/IBV-S2 was sequenced to determine any undesired mutation in the recombinant virus.

Biological Assessment of the NDV/IBV-S2 Recombinant Virus: Pathogenicity and Immunogenicity.

Replication and pathogenicity properties of the rLaSotailBV-S2 virus in embryonated chicken eggs and in chickens was evaluated and compared against the NDV LaSota strain (originally obtained from ATCC). Standard measurements included MDT (mean death time in embryonated eggs), ICPI (intracerebral pathogenicity index assay in day-old chickens), HA (hemagglutination activity), EID₅₀ (50% egg infective dose) and TCID₅₀ (50% tissue infectious dose assay in DF-1 cells). As seen in Table 2 both the original NDV LaSota and the recombinant NDV behaved similarly.

TABLE 2
Biological assessments of the NDV/IBV-S2 recombinant virus

| Virus | MDT^{a} | ICPI^{b} | HA^{c} | EID₅₀^{d} | TCID₅₀^{e} |
|---|---|---|---|---|---|
| LaSota | 110 hs | 0.15 | 1024 | 6.8 × 10⁸ | 3.5 × 10⁷ |
| rLS/IBV-S2 | 122 hs | 0 | 4096 | 1.76 × 10⁹ | 1.58 × 10⁸ |

^{a}MDT: Mean death time in embryonated eggs.
^{b}ICPI: Intracerebral pathogenicity index in day-old chickens.
^{c}HA: Hemagglutination titer.
^{d}EID₅₀: The 50% egg infective dose in embryonated eggs.
^{e}TCID₅₀: The 50% tissue infectious dose on DF-1 cells.

The S2 insert did not alter the biological properties of the vector. Furthermore, as seen in Table 3, the rNDV induced specific hemagglutination inhibition antibodies in vaccinated chickens and these chickens were protected against challenge with a lethal dose of NDV/CA02.

TABLE 3
Serum antibody response against NDV following vaccination and survival
of chickens after challenge with a lethal dose of NDV/CA02

| | Antibody response | | |
|---|---|---|---|
| Expt. | Seropositive birds | HI titer ^{a} | Survivors |
| PBS | 0/10 | 0 | 0/10 |
| rLS/IBV-S2 | 10/10 | 3.6 ± 1.6 | 10/10 |

^{a} Hemagglutination inhibition (HI) titer was expressed in log₂ of the mean ± standard deviation.

In summary, a recombinant NDV LaSota virus expressing the IBV S2 gene was produced. The recombinant virus was stable and neither the replication ability nor the pathogenicity of the rLaSota strain was altered as a result of the insert of the S2 gene.

Prime/Boost/Challenge Experiment 1.

We established 5 chicken Groups (n=15 each) in HEPA-filtered Horsfall-type isolation units and treated them as shown in Table 4.

TABLE 4
Experimental design

| | | Groups | | | | |
|---|---|---|---|---|---|---|
| | Age | 1 | 2 | 3 | 4 | 5 |
| Vaccination | 4 days | N/A | rLS/ | rLS/-E³ | rLS/-E | rLS/-E |
| | | | IBV-S2 | | | |
| Vaccination | 18 days | N/A | Mass² | Mass | | — |
| Challenge | 41 days | N/A | Ark³ | Ark | Ark | — |
| Evaluate | 3-5 d | ✓ | ✓ | ✓ | ✓ | ✓ |
| signs & | post- | | | | | |
| collect | challenge | | | | | |
| samples | | | | | | |

¹rLS/-E: Empty LaSota vector;
²Mass: Commercial IBV Massachusetts-type attenuated vaccine;
³Ark: IBV Arkansas virulent strain.
✓ indicates that these evaluations were performed.

Chickens in Group 2 were primed-vaccinated with rLS/IBV.S2 at 4 days of age and boosted with a commercial attenuated IBV Mass-serotype vaccine strain at 18 days of age. Chickens (n=12-16/group) were vaccinated with 100 μl of the recombinant virus stock containing 10⁷ EID₅₀/ml. Thus each chicken received 10⁶ EID₅₀/bird via intranasal/intraocular (IN/IO) routes. Booster vaccination was performed with a commercially available live-attenuated Massachusetts-type vaccine at the dose recommended by the vaccine manufacturer. The commercially available vaccine was delivered via the ocular route

Chickens were challenged with a virulent wild Ark IBV strain at 41 days of age. Mass-serotype vaccines have been observed to provide only limited protection against Ark-serotype strains, and the S1 amino acid sequences of the vaccine and challenge strains in this experiment were only 77% identical. Therefore, the challenge would indicate whether the vaccination protocol provided protection against heterologous virus. Control groups included chickens vaccinated with the empty vector (rLS/E)+Mass (Group 3), rLS/E only (Group 4), vaccinated with rLS/E only and challenged (Group 5), and unvaccinated/not challenged chickens (Group 1).

After challenge, the severity of respiratory signs was scored as follows: 1=normal; 2=respiratory rales detected at the examiner's ear; 3=respiratory rales detected at distance (without approaching the bird to the examiner's ear). The severity of respiratory signs was recorded for each bird and used along with the incidence (i.e., the number of birds with clinical signs/group) to calculate an index for each group.

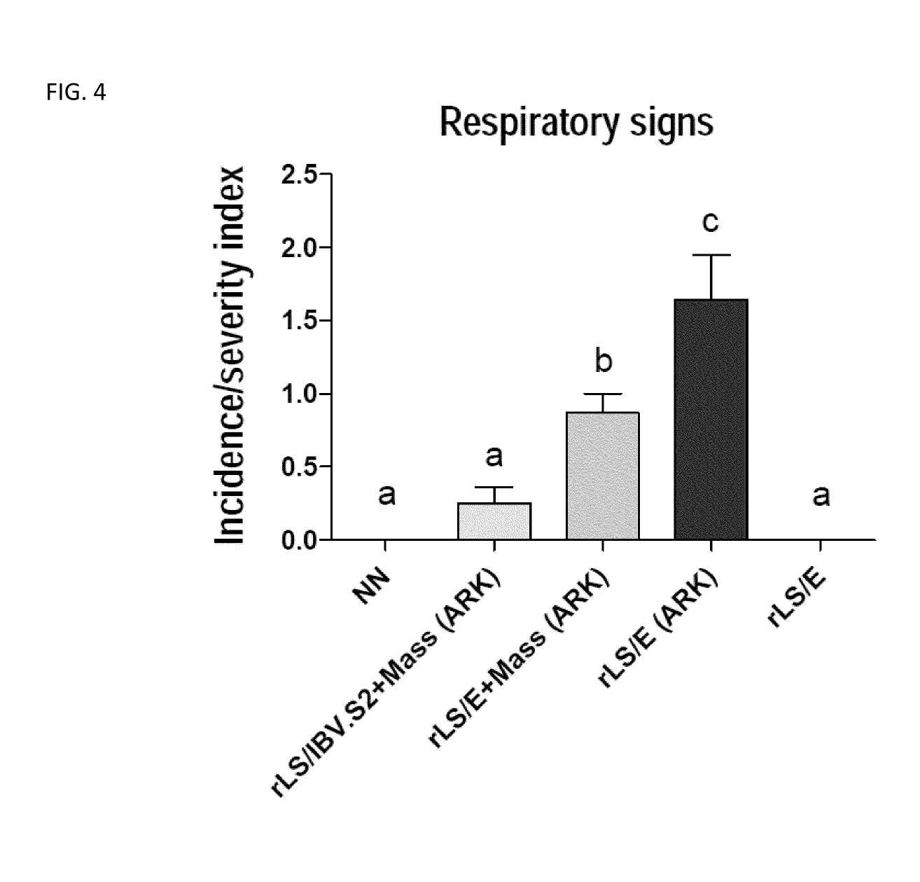

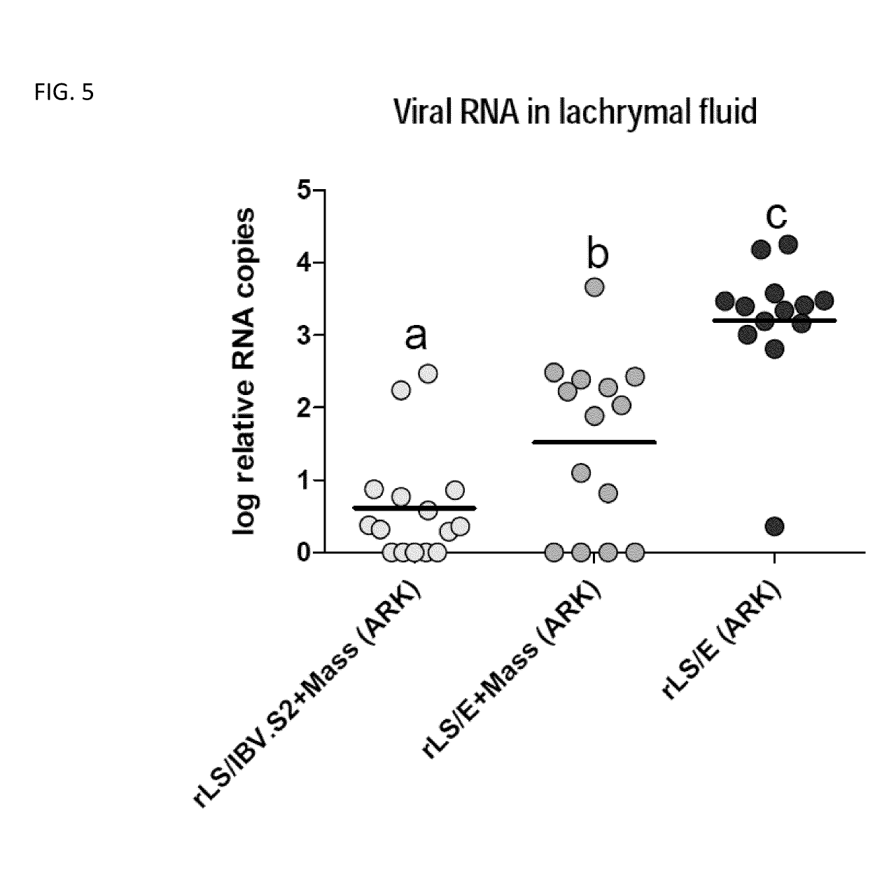

FIGS. 4 and 5 show the results obtained in the vaccination/challenge trial. Based on incidence and severity of clinical signs, chickens primed with rLS/IBV.S2 and boosted with an attenuated Mass-type vaccine were protected against challenge with a wild virulent Ark-type strain, (FIG. 4). In contrast, chickens vaccinated with the empty vector (rLS/E) showed significantly (P<0.05) higher incidence and severity of clinical signs. Indeed severe respiratory rales could be readily detected without approaching the individual birds to the ear of the examiner.

The group vaccinated with rLS/E+Mass attenuated vaccine (Group 3) showed significantly higher incidence and severity of respiratory signs than rLS/IBV.S2+Mass vaccinated chickens (Group 2) but significantly less signs than the positive control (rLS/E only, Group 5) (FIG. 4).

This result corroborated that Mass serotype IBV vaccination confers partial protection against Ark serotype IBV challenge. However, only by priming with rLS/IBV.S2 did the protection become complete, in that respiratory signs were not significantly different from unchallenged control birds. The results indicated by clinical signs were corroborated by the results of viral load detected in the lachrymal fluids of the challenged chickens at 4 days post-challenge (FIG. 5).

rLS/IBV.S2+Mass vaccinated chickens showed the lowest levels of IBV RNA of all groups. The reduction in viral load (as measured by qRT-PCR) was significant (P<0.05) compared to chickens vaccinated with rLS/E+Mass and chickens vaccinated with the empty vector only. Again the chickens vaccinated with Mass (and the empty vector) showed partial protection against challenge as determined by viral load.

### REFERENCES

Alexander, D. J., Senne, D. A., 2008. Newcastle Disease, Other Avian Paramyxoviruses, and Pneumovirus Infections, in: Saif, Y. M., Fadly, A. M., Glisson, J. R., McDougald, L. R., Nolan, L. K., Swayne, D. E. (Eds.), Diseases of Poultry. Blackwell Publishing, Ames, Iowa, USA, pp. 75-100.
Bowen, O., Estevez, C., Yu, Q., Suarez, D. L., 2010. Passive Antibody Transfer in Chickens to Model Maternal Antibody after Avian Influenza Vaccination, Annual Meeting of American Association of Avian Pathologists. Atlanta, Ga., p. 9216.
Bukreyev, A., Collins, P. L., 2008. Newcastle disease virus as a vaccine vector for humans. Current Opinion in Molecular Therapeutics 10, 46-55.
Bukreyev, A., Huang, Z., Yang, L., Elankumaran, S., St Claire, M., Murphy, B. R., Samal, S. K., Collins, P. L., 2005. Recombinant newcastle disease virus expressing a foreign viral antigen is attenuated and highly immunogenic in primates. J. Virol. 79, 13275-13284.
Cavanagh, D., 1981. Structural polypeptides of coronavirus IBV. J. Gen. Virol. 53, 93-103.
Cavanagh, D., 1983. Coronavirus IBV: structural characterization of the spike protein. J. Gen. Virol. 64, 2577-2583.
Cavanagh, D., 1984. Structural characterization of IBV glycoproteins. Adv. Exp. Med. Biol. 173, 95-108.
Cavanagh, D., Davis, P. J., 1986. Coronavirus IBV: removal of spike glycopolypeptide S1 by urea abolishes infectivity and haemagglutination but not attachment to cells. J. Gen. Virol. 67, 1443-1448.
Cavanagh, D., Davis, P. J., Darbyshire, J. H., Peters, R. W., 1986. Coronavirus IBV: Virus Retaining Spike Glycopolypeptide S2 but Not S1 Is Unable to Induce Virus-neutralizing or Haemagglutination-inhibiting Antibody, or Induce Chicken Tracheal Protection. J. Gen. Virol. 67, 1435-1442.
Corti, D., Jr., A. L. S., Pinna, D., Silacci, C., Fernandez-Rodriguez, B. M., Vanzetta, F., Santos, C., Luke, C. J., Torres-Velez, F. J., Temperton, N. J., Weiss, R. A., Sallusto, F., Subbarao, K., Lanzavecchia, A., 2010. Heterosubtypic neutralizing antibodies are produced by individuals immunized with a seasonal influenza vaccine. The Journal of Clinical Investigation 120, 1663-1673.
DiNapoli, J. M., Kotelkin, A., Yang, L., Elankumaran, S., Murphy, B. R., Samal, S. K., Collins, P. L., Bukreyev, A., 2007. Newcastle disease virus, a host range-restricted virus, as a vaccine vector for intranasal immunization against emerging pathogens. PNAS U.S.A. 104, 9788-9793.
DiNapoli, J. M., Ward, J. M., Cheng, L., Yang, L., Elankumaran, S., Murphy, B. R., Samal, S. K., Collins, P. L., Bukreyev, A., 2009. Delivery to the lower respiratory tract is required for effective immunization with Newcastle disease virus-vectored vaccines intended for humans. Vaccine 27, 1530-1539.
Enjuanes, L., Brian, D., Cavanagh, D., Holmes, K., Lai, M. M. C., Laude, H., Masters, P., Rottier, P., Siddell, S. G., Spaan, W. J. M., Taguchi, F., Talbot, P., 2000a. Coronaviridae, in: van Regenmortel, M. H. V., Fauquet, C. M., Bishop, D. H. L., Carstens, E. B., Estes, M. K., Lemon, S., Maniloff, J., Mayo, M., McGeoch, D. J., Pringle, C. R., Wickner, R. B. (Eds.), Virus taxonomy. Classification and nomenclature of viruses. Academic Press, New York, N.Y., pp. 835-849.
Enjuanes, L., Spaan, W. J., Snijder, E. J., Cavanagh, D., 2000b. Nidovirales, in: Regenmortel, M. H. V.v., Fauquet, C. M., Bishop, D. H. L., Carsten, E. B., Estes, M. K., Lemon, S. M., McGeoch, D. J., Maniloff, J., Mayo, M. A., Pringle, C. R., Wickner, R. B. (Eds.), Virus taxonomy. Classification and nomenclature of viruses Academic Press, New York, N.Y., pp. 827-834.
Estevez, C., King, D., Seal, B., Yu, Q., 2007. Evaluation of Newcastle disease virus chimeras expressing the Hemagglutinin-Neuraminidase protein of velogenic strains in the context of a mesogenic recombinant virus backbone. Virus Research 129, 182-190.
Estevez, C., King, D. J., Luo, M., Yu, Q., 2011. A single amino acid substitution in the hacmagglutinin-neuraminidase protein of Newcastle disease virus results in increased fusion promotion and decreased neuraminidase activities without changes in virus pathotype. J. Gen. Virol. 92, 544-551.
Fouchier, R. A. M., Munster, V., Wallensten, A., Bestebroer, T. M., Herfst, S.,
Smith, D., Rimmelzwaan, G. F., Olsen, B., Osterhaus, A. D. M. E., 2005. Characterization of a novel influenza A virus hemagglutinin subtype (H16) obtained from black-headed gulls. J. Virol. 79, 2814-2822.
Gallardo, R. A., Van Santen, V. L., Toro, H., 2010. Host intraspatial selection of infectious bronchitis virus populations. Avian Dis. 54, 807-813.
Ge, J., Deng, G., Wen, Z., Tian, G., Wang, Y., Shi, J., Wang, X., Li, Y., Hu, S., Jiang, Y., Yang, C., Yu, K., Bu, Z., Chen, H., 2007. Newcastle disease virus-based live attenuated vaccine completely protects chickens and mice from lethal challenge of homologous and heterologous H5N1 avian influenza viruses. J. Virol. 81, 150-158.
Ge, J., Tian, G., Zeng, X., Jiang, Y., Chen, H., Bua, Z., 2010. Generation and evaluation of a Newcastle disease virus-based H9 avian influenza live vaccine. Avian Dis. 54, 294-296.
Gelb, J., Jr., Keeler, C. L., Jr., Nix, W. A., Rosenberger, J. K., Cloud, S. S., 1997. Antigenic and S-1 genomic characterization of the Delaware variant serotype of infectious bronchitis virus. Avian diseases. 41, 661-669.
Hitchner, S. B., 2004. History of biological control of poultry diseases in the USA. Avian Dis. 48, 1-8.
Hu, H., Roth, J., Estevez, C. N., Zsak, L., Liu, B., Yu, Q., 2011. Generation and evaluation of a recombinant Newcastle disease virus expressing the glycoprotein (G) of avian metapneumovirus subgroup C as a bivalent vaccine in turkeys. Vaccine 29, 8624-8633.
Huang, Z., Elankumaran, S., Panda, A., Samal, S. K., 2003a. Recombinant Newcastle disease virus as a vaccine vector. Poultry Science 82, 899-906.
Huang, Z., Krishnamurthy, S., Panda, A., Samal, S. K., 2003b. Newcastle disease virus V protein is associated with viral pathogenesis and functions as an alpha interferon antagonist. J. Virol. 77, 8676-8685.
Huang, Z., Panda, A., Elankumaran, S., Govindarajan, D., Rockemann, D. D., Samal, S. K., 2004. The hemagglutinin-neuraminidase protein of Newcastle disease virus determines tropism and virulence. J. Virol. 78, 4176-4184.
Ignjatovic, J., Galli, U., 1995. Immune responses to structural proteins of avian infectious bronchitis virus. Avian Pathol. 24, 313-332.
Jackwood, M., 2012. Review of Infectious bronchitis virus around the world. Avian Dis. 56, 634-641.
Jackwood, M. W., Hilt, D. A., Lee, C. W., Kwon, H. M., Callison, S. A., Moore, K. M., Moscoso, H., Sellers, H., Thayer, S., 2005. Data from 11 years of molecular typing infectious bronchitis virus field isolates. Avian Dis. 49, 614-618.
Koch, G., Hartog, L., Kant, A., van Roozelaar, D. J., 1990. Antigenic domains on the peplomer protein of avian infectious bronchitis virus: correlation with biological functions. J. Gen. Virol. 71, 1929-1935.
Koch, G., Kant, A., 1990. Binding of antibodies that strongly neutralise intectious bronchitis virus is dependent on the glycosylation of the viral peplomer protein. Adv. Exp. Med. Biol. 276, 143-150.
Kusters, J. G., Jager, E. J., Lenstra, J. A., Koch, G., Posthumus, W. P., Meloen, R. H., van der Zeijst, B. A., 1989a. Analysis of an immunodominant region of infectious bronchitis virus. J. Immunol. 143, 2692-2698.
Kusters, J. G., Niesters, H. G., Bleumink-Pluym, N. M., Davelaar, F. G., Horzinek, M. C., van der Zeijst, B. A., 1987. Molecular epidemiology of infectious bronchitis virus in The Netherlands. J. Gen. Virol. 68, 343-352.
Kusters, J. G., Niesters, H. G. M., Lenstra, J. A., Horzinek, M. C., van der Zeijst, B. A. M., 1989b. Phylogeny of antigenic variants of avian coronavirus IBV. Virology 169, 217-221.
Lai, M. M. C., Cavanagh, D., 1997. The molecular biology of coronaviruses. Adv. Vir. Res. 48, 1-100.
Lai, M. M. C., Holmes, K. V., 2001. Coronaviridae: The viruses and their replication, in: Knipe, D. M., Howley, P. M. (Eds.), Fundamental Virology, 4th ed. Lippincott Williams & Wilkins, Philadelphia, pp. 641-663.
Lamb, R. A., Collins, P. L., Kolakofsky, D., Melero, J. A., Nagai, Y., Oldstone, M. B. A., Pringle, C. R., Rima, B. K., 2005. Family Paramyxoviridae, in: Fauquet, C. M., Mayo, M. A., Maniloff, J., Desselberger, U., Ball, L. A. (Eds.), Virus Taxonomy, Eighth Report of the International Committee on Taxonomy of Viruses. Elsevier Academic Press, San Diego, pp. 655-668.
Lenstra, J. A., Kusters, J. G., Koch, G., van der Zeijst, B. A., 1989. Antigenicity of the peplomer protein of infectious bronchitis virus. Molecular Immunology 26, 7-16.
McKinley, E. T., Hilt, D. A., Jackwood, M. W., 2008. Avian coronavirus infectious bronchitis attenuated live vaccines undergo selection of subpopulations and mutations following vaccination. Vaccine 26, 1274-1284.
Miller, P. J., Estevez, C., Yu, Q., Suarez, D. L., King, D. J., 2009. Comparison of viral shedding following vaccination with inactivated and live Newcastle disease vaccines formulated with wild-type and recombinant viruses. Avian Dis. 53, 39-49.
Mockett, A. P., Cavanagh, D., Brown, T. D., 1984. Monoclonal antibodies to the S1 spike and membrane proteins of avian infectious bronchitis coronavirus strain Massachusetts M41. J. Gen. Virol. 65, 2281-2286.
Nakaya, T., Cros, J., Park, M. S., Nakaya, Y., Zheng, H., Sagrera, A., Villar, E., Garcia-Sastre, A., Palese, P., 2001. Recombinant Newcastle disease virus as a vaccine vector. J. Virol. 75, 11868-11873.
Nayak, B., Rout, S. N., Kumar, S., Khalil, M. S., Fouda, M. M., Ahmed, L. E., Earhart, K. C., Perez, D. R., Collins, P. L., Samal, S. K., 2009. Immunization of chickens with Newcastle disease virus expressing H5 hemagglutinin protects against highly pathogenic H5N1 avian influenza viruses. PloS one 4, e6509.
Nix, W. A., Troeber, D. S., Kingham, B. F., Keeler, C. L., Jr., Gelb, J., Jr., 2000. Emergence of subtype strains of the Arkansas serotype of infectious bronchitis virus in Delmarva broiler chickens. Avian Dis. 44, 568-581.
Park, M. S., Steel, J., Garcia-Sastre, A., Swayne, D., Palese, P., 2006. Engineered viral vaccine constructs with dual specificity: avian influenza and Newcastle disease. PNAS U.S.A. 103, 8203-8208.
Pedersen, J. C., Senne, D. A., Woolcock, P. R., Kinde, H., King, D. J., Wise, M. G., Panigrahy, B., Seal, B. S., 2004. Phylogenetic relationships among virulent Newcastle disease virus isolates from the 2002-2003 outbreak in California and other recent outbreaks in North America. J. Clin. Microbiol. 42, 2329-2334.
Peeters, B. P., de Leeuw, O. S., Koch, G., Gielkens, A. L., 1999. Rescue of Newcastle disease virus from cloned cDNA: evidence that cleavability of the fusion protein is a major determinant for virulence. J. Virol. 73, 5001-5009.
Romer-Oberdorfer, A., Mundt. E., Mebatsion, T., Buchholz, U. J., Mettenleiter, T. C., 1999. Generation of recombinant lentogenic Newcastle disease virus from cDNA. J. Gen. Virol. 80, 2987-2995.
Shirato, K., Kawase, M., Watanabe, O., Hirokawa, C., Matsuyama, S., Nishimura, H., Taguchi, F., 2012. Differences in neutralizing antigenicity between laboratory and clinical isolates of HCoV-229E isolated in Japan in 2004 2008 depend on the S1 region sequence of the spike protein. J. Gen. Virol. 93, 1908-1917.
Souza, C. M., Rocha, F. R. T., Martins, N. R. S., Resende, J. S., M. A. Jorge, Rampinelli, A. P., 2001. Production of monoclonal antibodies against conserved components of infectious bronchitis virus. Arq. Bras. Med. Vet. Zootec. 53, 523-530.
Stadler, K., Masignani, V., Eickmann, M., Becker, S., Abrignani, S., Klenk, H.-D., Rappuoli, R., 2003. Comparison of coronavirus genome structures. Nature Rev. Microbiol. 1, 209-218.
Susta, L., Miller, P. J., Afonso, C. L., Estevez, C., Yu, Q., Zhang, J., Brown, C. C., 2010. Pathogenicity evaluation of different Newcastle disease virus chimeras in 4-week-old chickens. Trop Anim Health Prod 42, 1785-1795.
Swayne, D. E., Suarez, D. L., Schultz-Cherry, S., Tumpey, T. M., King, D. J., Nakaya, T., Palese, P., Garcia-Sastre, A., 2003. Recombinant paramyxovirus type 1-avian influenza-H7 virus as a vaccine for protection of chickens against influenza and Newcastle disease. Avian Dis. 47, 1047-1050.
Toro, H., Gallardo, R. A., Santen, V. L.v., Zhang, J. F., Joiner, K. S., 2012a. Protection induced by distinct IBV Ark-DPI S1 expressed from rAdenovirus, in: Lierz, M., Heffels-Redmann, U., Kaleta, E. F. (Eds.), VII. International Symposium on Avian Corona- and Pneumoviruses and Complicating Pathogens. World Veterinary Poultry Association, Rauischolzhausen, Germany, pp. 225-229.
Toro, H., Jackwood, J. W., Van Santen, V. L., 2012b. Genetic Diversity and Selection Regulates Evolution of Infectious Bronchitis Virus Avian Dis. 56, 449-455.
Toro, H., Pennington, D., R. A. Gallardo, Santen, V. L.v., Ginkel, F. W.v., Zhang, J. F., Joiner, K. S., 2012c. Infectious bronchitis virus subpopulations in vaccinated chickens after challenge Avian Dis. 56, 501-508.
Toro, H., Suarez, D. L., Tang, D., Ginkel, F.v., Shi, Z., 2007. RCA-free recombinant adenovirus-vectored vaccine for mass immunization of poultry against avian influenza, 2007 AAAP/AVMA Annual Meeting, Washington D.C.
Toro, H., Tang, D. C., Suarez, D. L., Zhang, J., Shi, Z., 2008. Protection of chickens against avian influenza with non-replicating adenovirus-vectored vaccine. Vaccine 26, 2640-2646.
Toro, H., van Santen, V. L., Li, L., Lockaby, S. B., van Santen, E., Hoerr, F. J., 2006. Epidemiological and experimental evidence for immunodeficiency affecting avian infectious bronchitis. Avian Pathol. 35, 1-10.
van Santen, V. L., Toro, H., 2008. Rapid selection in chickens of subpopulations within ArkDPI-derived infectious bronchitis virus vaccines. Avian Pathol. 37, 293-306.
Webster, R. G., Bean, W. J., Gorman, O. T., Chambers, T. M., Kawaoka, Y., 1992. Evolution and ecology of influenza A viruses. Microbiology Reviews 56, 152-179.
Wei, C. J., Boyington, J. C., McTamney, P. M., Kong, W. P., Pearce, M. B., Xu, L., Andersen, H., Rao, S., Tumpey, T. M., Yang, Z. Y., Nabel, G. J., 2010. Induction of broadly neutralizing H1N1 influenza antibodies by vaccination. Science 329, 1060-1064.
Yu, Q., Estevez, C., Song, M., Kapczynski, D., Zsak, L., 2010a. Generation and biological assessment of recombinant avian metapneumovirus subgroup C (aMPV-C) viruses containing different length of the G gene. Virus Research 147, 182-188.
Yu, Q., Estevez, C. N., Roth, J. P., Hu, H., Zsak, L., 2011. Deletion of the M2-2 gene from avian metapneumovirus subgroup C impairs virus replication and immunogenicity in turkeys. Virus Genes 42, 339-346.
Yu, Q., Roth, J., Hu, H., Estevez, C., Zsak. L., 2010b. Generation and characterization of recombinant Newcastle disease virus LaSota strain expressing avian metapneumovirus G protein, 26th Annual Meeting of American Society for Virology, p. 283.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of references are made herein. The cited references are incorporated by reference herein in their entireties. In the event that there is an inconsistency between a definition of a term in the specification as compared to a definition of the term in a cited reference, the term should be interpreted based on the definition in the specification.

1. A method for immunizing an avian against infectious bronchitis virus (IBV), the method comprising:
(a) administering a first composition comprising a viral vector that expresses an S2 polypeptide comprising the amino acid sequence of SEQ ID NO:1 or comprising an amino acid sequence having at least about 70% sequence identity to the amino acid sequence of SEQ ID NO:1; and
(b) administering a second composition comprising IBV.

2. The method of claim 1, wherein the first composition is administered in an amount that is effective for inducing an immune response against S2 polypeptide.

3. The method of claim 2, wherein the immune response is an antibody response.

4. The method of claim 2, wherein the immune response is a cell-mediated immune response.

5. The method of claim 1, wherein the second composition is administered in an amount that is effective for inducing an immune response against IBV.

6. The method of claim 5, wherein the immune response is an antibody response.

7. The method of claim 5, wherein the immune response is a cell-mediated immune response.

8. The method of claim 1, wherein the second composition is administered 1-4 weeks after administering the first composition.

9.-12. (canceled)

13. The method of claim 1, wherein the avian is a chicken.

14. The method of claim 1, wherein the second composition comprises an avirulent strain of IBV.

15. The method of claim 14, wherein the avirulent strain is an attenuated strain of IBV.

16. The method of claim 1, wherein the method provides heterotypic protection against IBV.

17. A kit for immunizing an avian against infectious bronchitis virus (IBV), the kit comprising:
(a) a first composition comprising a viral vector that expresses an S2 polypeptide comprising the amino acid sequence of SEQ ID NO:1 or comprising an amino acid sequence having at least about 70% sequence identity to the amino acid sequence of SEQ ID NO: 1; and
(b) a second composition comprising IBV.

18. The kit of claim 17, wherein the viral vector is selected from a group consisting of a paramyxovirus vector, a herpesvirus vector, and an adenovirus vector.

19.-21. (canceled)

22. The kit of claim 17, wherein the second composition comprises an avirulent strain of IBV.

23. The kit of claim 22, wherein the avirulent strain is an attenuated strain of IBV.

24. A paramyxovirus vector that expresses an S2 polypeptide comprising the amino acid sequence of SEQ ID NO:1 or comprising an amino acid sequence having at least about 70% sequence identity to the amino acid sequence of SEQ ID NO: 1.

25.-27. (canceled)

28. A vaccine composition comprising the paramyxovirus vector of claim 24.


SEQUENCE LISTING (2 sequences)

SEQ ID NO: 1 - protein, 625 aa, Avian Infectious Bronchitis Virus
SVTENVTNCPYVSYGKFCIKPDGSISVIVPKELDQFVAPLLNVTEYVLIPNSFNLTVTDEYIQTRMDKIQINCLQYVCGNSLACRKLFQQYGPVCDNILSVVNSVGQKEDMELLNFYSSTKPARFNTPVFSNLSTGEFNISLLLTPPSSPRRRSFIEDLLFTSVESVGLPTDDAYKKCTAGPLGFLKDLACAREYNGLLVLPPIITAEMQTLYTSSLVASMAFGGITATGAIPFATQLQARINHLGITQSLLVKNQEKIAASFNKAIGHMQEGFRSTSLALQQIQDVVNKQSAILTETMAALNKNFGAISSVIQDIYQQLDSIQADAQVDRLITGRLSSLSVLASAKQSEYIRVSQQRELATQKINECVKSQSIRYSFCGNGRHVLTIPQNAPNGIVFIHFTYTPESFINVTAIVGFCVSPANASQYAIVPANGRGIFIQVNGSYYITARDMYMPRDITAGDIVTLTSCQANYVSVNKTVITTFVDNDDFDFDDELSKWWNDTKHELPDFDKFNYTVPILDIDSEIDRIQGVIQGLNDSLIDLETLSILKTYIKWPWYVWVAIAFAPIIFILILGWVFFMTGCCGCCCGCFGIIPLMSKCGKKSSYYTTFDNDVVTEQYRPKKSV

SEQ ID NO: 2 - DNA, 1881 bp, Artificial
  Codon-optimized S2 gene of Avian Infectious Bronchitis Virus
atgagcgtgacagagaacgtgactaattgtccctacgtgtcctatggaaagttctgcatcaaaccagatgggagcatctccgtgattgtgcccaaggagctggaccagttcgtggcccctctgctgaacgtgactgaatacgtgctgatccctaactcctttaatctgacagtgactgatgaatacatccagacccggatggacaagatccagattaactgtctgcagtacgtgtgcggcaatagcctggcatgtagaaaactgtttcagcagtatggacccgtgtgcgataacatcctgtccgtggtgaacagcgtggggcagaaggaggacatggaactgctgaacttctacagctccactaaacccgcccggttcaacacccccgtgttcagcaatctgtccaccggagagtttaatatctccctgctgctgacacccccttctagccccagaaggcgctctttcattgaggatctgctgtttacatctgtggaaagcgtgggcctgccaacagatgacgcatacaagaaatgtactgccggccccctgggattcctgaaggacctggcttgcgcaagagagtacaacggactgctggtgctgccacccatcattaccgccgaaatgcagactctgtatacctcctctctggtggccagcatggctttcggcggaatcaccgcaacaggggccattccctttgccacacagctgcaggctaggatcaaccacctgggcattactcagtctctgctggtgaagaaccaggagaaaatcgccgccagcttcaacaaggctattgggcacatgcaggaaggctttcgctctacaagcctggctctgcagcagatccaggatgtggtgaataagcagtccgcaattctgactgagacaatggccgccctgaacaagaatttcggagcaatcagctccgtgatccaggatatctaccagcagctggacagcatccaggccgatgctcaggtggaccggctgattaccgggagactgtctagcctgagcgtgctggcatccgccaagcagtctgaatacatcagggtgagccagcagcgcgagctggctacacagaagatcaacgaatgcgtgaaatcccagtctattaggtattccttctgcgggaatggccgccacgtgctgactatccctcagaacgccccaaatggcatcgtgttcattcattttacatacactccagagagcttcatcaacgtgaccgctattgtgggattttgcgtgagcccagctaatgcatcccagtatgctatcgtgcccgcaaacggaagggggatcttcattcaagtgaatggaagctactatattactgctcgggacatgtacatgcccagagatatcaccgcaggggacattgtgaccctgacatcttgccaggccaactatgtgagcgtgaataagacagtgatcaccacatttgtggataacgatgacttcgactttgatgacgagctgtccaagtggtggaacgataccaaacacgaactgcctgatttcgacaagttcaactacacagtgccaatcctggatattgactctgagatcgacaggattcagggcgtgatccagggactgaatgattccctgattgacctggaaaccctgtctatcctgaagacatacatcaagtggccctggtacgtgtgggtggccatcgctttcgcacctatcatttttatcctgattctggggtgggtgttctttatgacagggtgctgtggctgctgttgcggatgtttcgggatcattcctctgatgtctaagtgcggcaagaaatcctcttactatactacctttgataacgacgtggtgactgagcagtaccgcccaaagaaaagcgtgtga